(19) United States
(12) Patent Application Publication
(10) Pub. No.: US 2014/0272608 A1
(43) Pub. Date: Sep. 18, 2014

(54) MULTICOPPER OXIDASE MUTANT, A GENE CODING THEREOF, AND A BIOFUEL-CELL USING THE SAME

(71) Applicant: TOYOTA JIDOSHA KABUSHIKI KAISHA, Toyota-shi, Aichi-ken (JP)

(72) Inventors: Takahiro Kusumegi, Toyota-shi (JP); Kumi Terada, Toyota-shi (JP)

(21) Appl. No.: 14/349,777

(22) PCT Filed: Oct. 5, 2012
(86) PCT No.: PCT/JP2012/006424

§ 371 Date: Apr. 4, 2014

(30) Foreign Application Priority Data
Oct. 7, 2011 (JP) 2011-222756

(51) Int. Cl. H01M 8/16; C12N 9/02

(52) U.S. Cl. CPC H01M 8/16; C12N 9/001
USPC 429/401; 435/189; 536/23.2

(57) ABSTRACT

A multicopper oxidase mutant having improved resistance to imidazole compounds is provided. A multicopper oxidase mutant, which comprises an amino acid sequence derived from the amino acid sequence shown in SEQ ID NO: 2 by substitution of either or both an amino acid residue corresponding to methionine at position 157 and an amino acid residue corresponding to proline at position 414 with a different amino acid and has activity of catalyzing a reaction generating water molecules via four-electron reduction of oxygen molecules using ABTS (2,2′-azinobis(3-ethylbenzoline-6-sulfonate)) as a substrate is provided.

### TECHNICAL FIELD

The present invention relates to a multicopper oxidase mutant having a substitution mutation at a position corresponding to a given position in a multicopper oxidase, a gene encoding such multicopper oxidase mutant, and a biofuel cell using the multicopper oxidase mutant.

### BACKGROUND ART

Multicopper oxidases (bilirubin oxidases) are proteins that have the four copper atoms necessary for enzyme activity within the molecules. They serve as oxidoreductases for catalyzing a reaction generating water molecules via four-electron reduction of oxygen molecules using electrons removed from an arbitrary substrate. As described in Patent Literature 1 to 4, multicopper oxidases are used for cathode electrodes of biofuel cells or used as electrode materials for a variety of biosensors. In addition, as suggested in Patent Literature 1 to 3, there have been attempts to introduce at least one amino acid substitution mutation into a multicopper oxidase so as to modify the functions of the enzyme in order to prevent reduction of enzyme activity during immobilization, improve thermostability of the enzyme, or reduce the reaction overpotential.

Biofuel cells are also referred to as “enzyme fuel cells” in which electrical energy is generated in a chemical reaction caused by an enzyme for use of electrical energy. As in cases of conventional batteries, biofuel cells have structures in which a cathode electrode and an anode electrode face each other separated by an electrolyte, and alcohol (e.g., methanol or ethanol) or sugar (e.g., glucose) is used as fuel. In addition, as described in Patent Literature 5, it is known that imidazole compounds can be used as electrolytes.

However, when an imidazole compound is used as an electrolyte, the compound causes degeneration of an enzyme (e.g., the above multicopper oxidase) used in a biofuel cell, resulting in reduction of enzyme activity. As shown in Patent Literature 5, there are examples of use an enzyme in the presence of imidazole compounds. However, it can be expected that the enzyme may not exhibit sufficient activity. The output of the fuel cells also depends on various factors except for the enzyme. Therefore, development of an enzyme having resistance to imidazole compounds improves the output of the fuel cells.

### CITATION LIST

### Patent Literature

PTL 1: JP Patent Publication (Kokai) No. 2009-158480 A

PTL 2: JP Patent Publication (Kokai) No. 2008-161178 A

PTL 3: JP Patent Publication (Kokai) No. 2010-183857 A

PTL 4: JP Patent Publication (Kokai) No. 2009-044997 A

PTL 5: JP Patent Publication (Kokai) No. 2009-158458 A

### SUMMARY OF INVENTION

### Technical Problem

In view of the above circumstances, an object of the present invention is to provide a multicopper oxidase mutant for which reduction of enzyme activity can be prevented even in the presence of imidazole compounds; that is to say, a multicopper oxidase mutant having improved resistance to imidazole compounds. Another object of the present invention is to provide a gene encoding such multicopper oxidase and a biofuel cell using the same.

### Solution to Problem

As a result of intensive studies to achieve the above object, the present inventors have found that a substitution mutation of an amino acid at a given position in a multicopper oxidase allows prevention of reduction in multicopper oxidase activity caused by imidazole compounds, making it possible to remarkably improve resistance of the multicopper oxidase to the imidazole compounds. This has led to the completion of the present invention.

Specifically, the multicopper oxidase mutant of the present invention comprises an amino acid sequence derived from the amino acid sequence shown in SEQ ID NO: 2 by substitution of either or both an amino acid residue corresponding to methionine at position 157 and an amino acid residue corresponding to proline at position 414 with a different amino acid and has activity of catalyzing a reaction generating water molecules via four-electron reduction of oxygen molecules using ABTS (2,2′-azinobis(3-ethylbenzoline-6-sulfonate)) as a substrate.

In addition, the amino acid residue corresponding to methionine at position 157 is preferably substituted with leucine and the amino acid residue corresponding to proline at position 414 is preferably substituted with leucine or threonine in the multicopper oxidase mutant of the present invention.

More preferably, an amino acid residue corresponding to histidine at position 90 is further substituted with a different amino acid in the amino acid sequence of the multicopper oxidase mutant of the present invention in which an amino acid residue corresponding to methionine at position 157 has been substituted with a different amino acid. In such case, it is particularly preferable for an amino acid residue corresponding to histidine at position 90 to be substituted with arginine.

Moreover, in the most preferable embodiment of the multicopper oxidase mutant of the present invention, the amino acid residue corresponding to methionine at position 157 is substituted with a different amino acid, the amino acid residue corresponding to proline at position 414 is substituted with a different amino acid, and the amino acid residue corresponding to histidine at position 90 is substituted with a different amino acid. Particularly preferably, in such case, the amino acid residue corresponding to methionine at position 157 is substituted with leucine, the amino acid residue corresponding to proline at position 414 is substituted with leucine, and the amino acid residue corresponding to histidine at position 90 is substituted with arginine.

Further, the multicopper oxidase mutant gene of the present invention comprises a polynucleotide encoding the above multicopper oxidase mutant. Specifically, the multicopper oxidase mutant gene of the present invention encodes a protein which comprises an amino acid sequence derived from the amino acid sequence shown in SEQ ID NO: 2 by substitution of either or both an amino acid residue corresponding to methionine at position 157 and an amino acid residue corresponding to proline at position 414 with a different amino acid and has activity of catalyzing a reaction generating water molecules via four-electron reduction of oxygen molecules using ABTS (2,2′-azinobis(3-ethylbenzoline-6-sulfonate)) as a substrate.

Furthermore, the above multicopper oxidase mutant is used for a cathode electrode in the biofuel cell of the present invention. Specifically, the biofuel cell of the present invention has a structure in which a cathode and an anodeface each other separated by an electrolyte and uses the multicopper oxidase mutant of the present invention as a catalyst for the positive electrode. Here, the multicopper oxidase mutant can be immobilized to an electrode by a conventionally known technique. In addition, an electrolyte used herein preferably comprises imidazole compound(s).

### Advantageous Effects of Invention

The multicopper oxidase mutant of the present invention has a novel substitution mutation and thus has significantly improved resistance to imidazole compounds over the corresponding unmutated multicopper oxidase. Reduction of multicopper oxidase activity can be prevented even in the presence of imidazole compounds in a biofuel cell using the multicopper oxidase mutant of the present invention. This allows long-term maintenance of excellent battery characteristics.

### BRIEF DESCRIPTION OF DRAWINGS

FIG. 1 is a characteristic diagram showing results of a comparison of amino acid sequences of conventionally known multicopper oxidases for which the present invention can be used.

FIG. 2 is a characteristic diagram showing residual activity levels of multicopper oxidase mutants treated with imidazole compounds.

FIG. 3 is a characteristic diagram showing specific activity levels of multicopper oxidase mutants.

### DESCRIPTION OF EMBODIMENTS

The present invention is described in detail with reference to the drawings.

<Multicopper Oxidase>

The multicopper oxidase mutant of the present invention comprises an amino acid sequence obtained by substituting a given amino acid residue with a different amino acid in a multicopper oxidase. Here, the multicopper oxidase is not particularly limited as long as it is a protein having the four copper atoms necessary for enzyme activity within the molecule and has the activity of catalyzing a reaction generating water molecules via four-electron reduction of oxygen molecules using electrons removed from an arbitrary substrate (hereinafter referred to as multicopper oxidase activity). Multicopper oxidases use a variety of substances as substrates that allow them to exhibit the above multicopper oxidase activity. An example of such a substrate is ABTS (2,2′-azinobis(3-ethylbenzoline-6-sulfonate)). In this specification, the term “multicopper oxidase activity” may refer to activity of catalyzing a reaction generating water molecules via four-electron reduction of oxygen molecules with electrons removed from ABTS. Another example of the substrate is bilirubin. In addition, a multicopper oxidase that has the activity of catalyzing a reaction generating two biliverdin molecules and water molecules from bilirubin and oxygen molecules using bilirubin as a substrate is referred to as “bilirubin oxidase.”

Examples of other substrates that can be used include oxidoreductive organic or inorganic compounds such as ferrocene, ferricyanide-alkaline metals (e.g., potassium ferricyanide, lithium ferricyanide, and sodium ferricyanide) or alkyl substitutes thereof (e.g., methyl substitute, ethyl substitute, and propyl substitute), phenazine methosulfate, p-benzoquinone, 2,6-dichlorophenolindophenol, methylene blue, beta-naphthoquinone-4-potassium sulfonate, phenazine ethosulfate, vitamin K, viologen, and Os complexes (e.g., the Os complexes described in JP Patent Publication (Kohyo) No. 2003-514823 A and JP Patent Publication (Kohyo) No. 2003-514924 A). In addition to the above, examples of substrates include: metal complexes mainly comprising metal elements such as Os, Fe, Ru, Co, Cu, Ni, V, Mo, Cr, Mn, Pt, and W or metal ions thereof; quinones such as quinone, benzoquinone, anthraquinone, and naphthoquinone; and heterocyclic compounds such as viologen, methylviologen, and benzylviologen. Further, a variety of compounds described as electron transfer mediators immobilized to a cathode (positive electrode) in JP Patent Publication (Kokai) No. 2011-124090 A and JP Patent Publication (Kokai) No. 2009-245930 A can be used as substrates.

A multicopper oxidase may be a plant-derived enzyme, an animal-derived enzyme, or a microorganism-derived enzyme. Examples of a microorganism-derived multicopper oxidase include a Bacillus subtilis-derived multicopper oxidase and a Myrothecium verrucaria-derived multicopper oxidase.

The gene nucleotide sequence of a Bacillus subtilis-derived multicopper oxidase and the amino acid sequence of a multicopper oxidase encoded by the gene are shown in SEQ ID NOS: 1 and 2, respectively. In addition, the amino acid sequence of a multicopper oxidase encoded by a Myrothecium verrucaria-derived multicopper oxidase gene is shown in SEQ ID NO: 3. An N-terminal-deficient-Myrothecium-verrucaria-derived multicopper oxidase is disclosed with accession no: 3ABC_B in a known sequence database. The amino acid sequence of the Myrothecium verrucaria-derived multicopper oxidase with accession no. 3ABC_B is shown in SEQ ID NO: 4.

A multicopper oxidase that can be used in the present invention is not particularly limited to a multicopper oxidase comprising the amino acid sequence shown in SEQ ID NO: 2, 3, or 4. For example, it may be a multicopper oxidase comprising an amino acid sequence derived from the amino acid sequence shown in SEQ ID NO: 2, 3, or 4 by deletion, substitution, addition, or insertion of one or more amino acids (other than amino acid residues to be substituted described in detail below) and having multicopper oxidase activity. Here, the term “one or more amino acids” refers to, for example, 1 to 30 amino acids, preferably 1 to 20 amino acids, more preferably 1 to 10 amino acids, further preferably 1 to 5 amino acids, and particularly preferably 1 to 3 amino acids. Deletion, substitution, or addition of amino acids can be carried out by modifying a gene encoding the multicopper oxidase by a method known in the art. For gene mutation, a conventionally known method such as the Kunkel method or the Gapped duplex method or a method in accordance therewith can be used. For example, a mutagenesis kit (e.g., Mutant-K or Mutant-G (product name; TAKARA)) using a site-specific mutation induction method or an LA PCR in vitro Mutagenesis series kit (product name; TAKARA) can be used for mutagenesis.

In addition, according to the present invention, a protein which comprises an amino acid sequence having, for example, 85% or more, preferably 90% or more, and more preferably 95% or more, and most preferably 98% or more sequence similarity to the amino acid sequence shown in SEQ ID NO: 2, 3, or 4 and has multicopper oxidase activity can be used as a multicopper oxidase. Here, the value of sequence similarity refers to a value that can be found based on default setting using a computer program equipped with a BLAST algorithm.

Further, a protein, which has multicopper oxidase activity and is encoded by a polynucleotide that hybridizes under stringent conditions to a polynucleotide complementary to a part or the whole of the nucleotide sequence shown in SEQ ID NO: 1, can be used as a multicopper oxidase in the present invention. Here, hybridization under stringent conditions means binding that is maintained during washing with 2×SSC at 60 degrees C. Hybridization can be carried out by a conventionally known method such as the method described in J. Sambrook et al. Molecular Cloning, A Laboratory Manual, 2nd Ed., Cold Spring Harbor Laboratory (1989).

A multicopper oxidase used herein is not limited to the above Bacillus-subtilis-derived or Myrothecium-verrucaria-derived multicopper oxidase. A multicopper oxidase from any organism species can be used in the present invention. For example, the amino acid sequences of multicopper oxidases from various types of species can be identified by searching a database containing gene information.

According to the present invention, any term can be used instead of the term “multicopper oxidase” for an enzyme as long as the enzyme has the above activity. For example, a known term such as laccase, bilirubin oxidase, multicopper oxidase, or blue copper oxidase can be used.

<Substitution Mutation>

The multicopper oxidase mutant of the present invention is obtained by substituting a given amino acid residue of the amino acid sequence of any of the above multicopper oxidases from various types of organism species such that it has resistance to imidazole compounds, which is significantly improved more than that of the multicopper oxidase before amino acid substitution. Here, an amino acid residue to be substituted can be identified with a numeral determined by reckoning the number of amino acid residues from the N terminus of Bacillus subtilis-derived multicopper oxidase comprising the amino acid sequence shown in SEQ ID NO: 2. However, the specific numeral for an amino acid residue to be substituted that is identified based on the amino acid sequence shown in SEQ ID NO: 2 would vary depending on the multicopper oxidase type. Therefore, “an amino acid residue at position X in the amino acid sequence shown in SEQ ID NO: 2” does not correspond to an amino acid residue at position X in a multicopper oxidase comprising an amino acid sequence that differs from the amino acid sequence shown in SEQ ID NO: 2, resulting in a different numeral for an amino acid residue to be substituted.

In the case of an amino acid sequence that differs from the amino acid sequence shown in SEQ ID NO: 2, an amino acid residue which corresponds to a given amino acid residue in the amino acid sequence shown in SEQ ID NO: 2 can be identified by multiple alignment analysis of a plurality of amino acid sequences, including the amino acid sequence shown in SEQ ID NO: 2. Multiple alignment analysis is not particularly limited. A person skilled in the art can readily carry out multiple alignment analysis using the CLUSTAL W (1.83) multiple sequence alignment program (available at the National Institute of Genetics (NIC) for DDBJ (http://clustalw.ddbj.nig.ac.jp/top-j.html)). If pairwise alignment analysis is used for alignment of the amino acid sequence shown in SEQ ID NO: 2 and a different amino acid sequence, an amino acid residue that corresponds to a given amino acid residue in the amino acid sequence shown in SEQ ID NO: 2 can be identified in the different amino acid sequence.

FIG. 1 shows the results of multiple alignment analysis for a Bacillus subtilis-derived multicopper oxidase (SEQ ID NO: 2) and Myrothecium verrucaria-derived multicopper oxidases (SEQ ID NOS: 3 and 4). In the multiple alignment shown in FIG. 1, the 1st and 2nd lines show the Myrothecium verrucaria-derived multicopper oxidases, and the 3rd line shows the Bacillus subtilis-derived multicopper oxidase. In addition to such specific multicopper oxidases, other multicopper oxidases can be subjected to multiple alignment analysis. The position of a given amino acid residue can be identified based on the Bacillus subtilis-derived multicopper oxidase (SEQ ID NO: 2). Hereinafter, an amino acid to be substituted is described based on the amino acid sequence shown in SEQ ID NO: 2; that is to say, the amino acid sequence of the Bacillus subtilis-derived multicopper oxidase. Note that a numeral that denotes the position of an amino acid would vary depending on the multicopper oxidase type, as described above. The multicopper oxidase mutant of the present invention includes a multicopper oxidase mutant that has a substitution mutation of an amino acid residue and a multicopper oxidase mutant derived from such multicopper oxidase mutant by further substitution mutation of an amino acid residue, as described below.

<Multicopper Oxidase Mutant>

The multicopper oxidase mutant of the present invention comprises an amino acid sequence derived from the amino acid sequence shown in SEQ ID NO: 2 by substitution of either or both an amino acid residue corresponding to methionine at position 157 and an amino acid residue corresponding to proline at position 414 with a different amino acid. In FIG. 1, an amino acid residue corresponding to methionine at position 157 and an amino acid residue corresponding to proline at position 414 are boxed. FIG. 1 shows that methionine at position 157 in the amino acid sequence shown in SEQ ID NO: 2 is also conserved in the Myrothecium verrucaria-derived multicopper oxidase.

Here, a different amino acid is not particularly limited. It can be any amino acid as long as a multicopper oxidase mutant has significantly improved resistance to imidazole compounds over the corresponding unmutated multicopper oxidase. The resistance to imidazole compounds can be evaluated based on residual activity determined after treatment (e.g., at 90 degrees C. for 30 minutes) in a solution containing imidazole compound(s) for a certain period of time. In addition, the improvement of resistance to imidazole compounds indicates that the residual activity of a multicopper oxidase mutant is statistically significantly greater than that of the unmutated wild-type multicopper oxidase. Enzyme activity of a multicopper oxidase mutant or that of the corresponding unmutated (unsubstituted) multicopper oxidase can be adequately determined by a conventionally known method. For instance, the residual activity of a multicopper oxidase can be determined by reacting the multicopper oxidase in a pH-adjusted buffer solution comprising 2,2′-azinobis(3-ethylbenzothiazoline-6-sulfonic acid) (ABTS) ammonium salt as a substrate and determining changes in the absorbance of the reaction product of ABTS. Accordingly, it can be determined whether or not substitution mutation of a given amino acid is effective for improving resistance to imidazole compounds.

More specifically, regarding the above substitution mutation, it is particularly preferable for the amino acid residue corresponding to methionine at position 157 and the amino acid residue corresponding to proline at position 414 to be substituted with leucine and leucine or threonine, respectively. In addition, a multicopper oxidase mutant may have either or both the substitution mutation of methionine at position 157 and the substitution mutation of proline at position 414. In either case, the multicopper oxidase mutant has higher resistance to imidazole compounds than the corresponding unmutated multicopper oxidase.

More preferably, an amino acid residue corresponding to histidine at position 90 is further substituted with a different amino acid in the amino acid sequence of the multicopper oxidase mutant of the present invention, in which an amino acid residue corresponding to methionine at position 157 has been substituted with a different amino acid. Particularly preferably, the amino acid residue corresponding to histidine at position 90 is substituted with arginine. In addition, a multicopper oxidase mutant that has a substitution mutation of histidine at position 90 alone has resistance to imidazole compounds comparable to that of the wild-type multicopper oxidase. However, such substitution mutation of histidine at position 90 enhances the resistance to imidazole compounds improved as a result of the above substitution mutation of methionine at position 157. Specifically, a multicopper oxidase double-mutant that has a substitution mutation of methionine at position 157 and a substitution mutation of histidine at position 90 has remarkably improved resistance to imidazole compounds over a multicopper oxidase mutant that has a substitution mutation of methionine at position 157 alone.

Specific examples of preferable amino acids serving as substituents are described above. However, such amino acids serving as substituents are not limited to the above examples. As described in Reference (1) (“McKee's Biochemistry,” Third Edition, Chapter 5, Amino acid, Peptide, Protein, 5.1 Amino acid, Atsushi Ichikawa (editor), Shinichi Fukuoka (translator), Ryosuke Sone (publisher), published by Kagaku-Dojin Publishing Co., Inc., ISBN4-7598-0944-9)), it is well-known that amino acids are classified in accordance with side chains having similar properties (i.e., chemical properties or physical size). Also, it is well-known that, in molecular evolution, substitution of amino acid residues, which are classified into a given group, takes place at high frequency while protein activity is retained. Based on such concept, Reference (2) (Henikoff S., Henikoff J. G., Amino-acid substitution matrices from protein blocks, Proc. Natl. Acad. Sci. USA, 89, 10915-10919 (1992)) proposes in FIG. 2 that the amino acid residue substitution scoring matrix (BLOSUM), and this is extensively used. Reference (2) is based on the principle that substitution of amino acids having similar side chain chemical properties imposes little influence on proteins in terms of structural or functional changes. According to References (1) and (2), side chain groups of amino acids in terms of the multiple alignment can be determined based on indicators such as chemical properties and physical sizes. This is indicated as a group of amino acids having a score of 0 or larger and preferably as a group of amino acids having a score of 1 or larger in the scoring matrix (BLOSUM) disclosed in Reference (2).

Based on the above findings, amino acids having similar properties can be classified as a member of one of the eight groups described below. Therefore, after substitution, each amino acid is preferably classified as a member of the group that includes the corresponding amino acid described above. For example, methionine at position 157 in a Bacillus subtilis-derived multicopper oxidase is preferably substituted with leucine. Alternatively, the methionine residue may be substituted with isoleucine, methionine, or valine, which is classified as a member of the following group, of which leucine is also a member: 1) Group of hydrophobic aliphatic amino acids. Similarly, proline at position 414 in a Bacillus subtilis-derived multicopper oxidase is preferably substituted with leucine or threonine. Alternatively, the proline residue may be substituted with isoleucine, methionine, or valine, which is classified as a member of the following group, of which leucine is also a member: 1) Group of hydrophobic aliphatic amino acids. Or, it may be substituted with serine, which is classified as a member of the following group, of which threonine is also a member: 2) Group having hydroxymethylene groups. Further, histidine at position 90 in a Bacillus subtilis-derived multicopper oxidase is preferably substituted with arginine. Alternatively, the histidine residue may be substituted with lysine, which is classified as a member of the following group, of which arginine is also a member: 4) Group of basic amino acids.

1) Group of Hydrophobic Aliphatic Amino Acids (ILMV Group)

This is a group of amino acids having hydrophobic aliphatic side chains selected from among the neutral and non-polar amino acids described in Reference (1), which is composed of V (Val, valine), L (Leu, leucine), I (Ile, isoleucine), and M (Met, methionine). Among amino acids that are classified as neutral and non-polar amino acids according to Reference (1), FGACWP is not included in “the group of hydrophobic aliphatic amino acids” for the following reasons. That is, G (Gly, glycine) and A (Ala, alanine) are smaller than methyl groups and have small non-polar effects. Also, C (Cys, cysteine) occasionally plays a key role in S—S bonds and forms a hydrogen bond with an oxygen atom or nitrogen atom. Further, side chains of F (Phe, phenylalanine) and W (Trp, tryptophan) have very large molecular weights and have potent aromatic effects. Also, P (Pro, proline) has potent imino acid effects and disadvantageously immobilizes the angle of the polypeptide main chain.

2) Group Having Hydroxymethylene Groups (ST Group)

This is a group of amino acids having hydroxymethylene groups on the side chains selected from among the neutral and polar amino acids, which is composed of S (Ser, serine) and T (Thr, threonine). Since hydroxyl groups that are present on S and T side chains are sugar-binding sites, such hydroxyl groups often serve as important sites allowing a given polypeptide (a protein) to have a given activity.

3) Group of Acidic Amino Acids (DE Group)

This is a group of amino acids having acidic carboxyl groups on the side chains, which is composed of D (Asp, aspartic acid) and E (Glu, glutamic acid).

4) Group of Basic Amino Acids (KR Group)

This is a group of basic amino acids, which is composed of K (Lys, lysine) and R (Arg, arginine). K and R positively charge over a wide pH range and have basic properties. H (His, histidine), which is classified as a basic amino acid, is not substantially ionized at pH 7, and thus it is not classified as a member of this group.

5) Group of Amino Acids Having Methylene Groups or Polar Groups (DHN Group)

Amino acids of this group have carbon atoms at the alpha positions, methylene groups bound thereto as side chains, and polar groups at farther positions. Physical sizes of non-polar methylene groups are very similar, and the group is composed of N (Asn, asparagine; an amide group as a polar group), D (Asp, aspartic acid; a carboxyl group as a polar group), and H (His, histidine; an imidazole group as a polar group).

6) Group of Amino Acids Having Dimethylene Groups or Polar Groups (EKQR Group)

Amino acids of this group have carbon atoms at the alpha positions, linear hydrocarbons of dimethylene or higher bound thereto as side chains, and polar groups at farther positions. Physical sizes of non-polar dimethylene groups are very similar, and such groups are composed of E (Glu, glutamic acid; a carboxyl group as a polar group), K (Lys, lysine; an amino group as a polar group), Q (Gln, glutamine; an amide group as a polar group), and R (Arg, arginine, imino and amino groups as polar groups).

7) Group of Aromatic Amino Acids (FYW Group)

This is a group of aromatic amino acids having benzene nuclei on the side chains, and this group has chemical properties peculiar to aromatic amino acids. The group is composed of F (Phe, phenylalanine), Y (Tyr, tyrosine), and W (Trp, tryptophan).

8) Group of Cyclic and Polar Amino Acids (HY Group)

This group is composed of amino acids simultaneously having a cyclic structure and a polar group on the side chains. The group is composed of H (H, histidine; the cyclic structure and the polar group are imidazole groups), Y (Tyr, tyrosine; the cyclic structure is the benzene nucleus and the polar group is a hydroxyl group).

<Production of the Multicopper Oxidase Mutant>

The above multicopper oxidase mutant of the present invention can be obtained by a conventionally known protein production method. For example, a eukaryote-derived multicopper oxidase mutant can be obtained by a protein production system using yeast as a host. Also, a prokaryote-derived multicopper oxidase mutant can be obtained by a protein production system using Escherichia coli as a host or a cell-free protein production system.

More specifically, a gene encoding the above multicopper oxidase mutant is prepared as described below. For instance, a gene encoding a multicopper oxidase mutant can be prepared via mutagenesis at a given site in the wild-type multicopper oxidase gene in accordance with the site-specific mutagenesis method of T. Kunkel (Kunkel, T. A. Proc. Nati. Acad. Sci. USA, 82, 488-492 (1985)), the Gapped duplex method, or the like. Alternatively, a gene encoding the above multicopper oxidase mutant can be prepared by subjecting, for example, the wild-type multicopper oxidase to mutagenesis using a mutagenesis kit by a site-specific mutagenesis method (e.g., Mutan-K (Takara Shuzo Co., Ltd.) or Mutan-G (Takara Shuzo Co., Ltd.)) or using an LA PCR in vitro Mutagenesis series kit (Takara Shuzo Co., Ltd.).

Particularly preferably, a Bacillus subtilis-derived multicopper oxidase mutant is produced as the multicopper oxidase mutant of the present invention. This is because the degree of heat resistance of a Bacillus subtilis-derived multicopper oxidase is much higher than that of any other multicopper oxidase. In addition, a prokaryote-derived multicopper oxidase (such as a Bacillus subtilis-derived multicopper oxidase) differs from a eukaryote-derived multicopper oxidase in that there is no need to carry out sugar chain modification or the like for a prokaryote-derived multicopper oxidase. Thus, a Bacillus subtilis-derived multicopper oxidase is preferable because it can be readily produced by a protein production system using Escherichia coli or a cell-free protein production system.

When a protein production system in which yeast serves as a host is used, a conventionally used expression vector comprising a multicopper oxidase mutant gene can be introduced into yeast. Typically, a vector has selection marker genes, cloning sites, and expression control regions (promoters and terminators). Such vector is well known in the art and commercially available. Promoters contained in a vector may be constitutive expression promoters or inducible promoters, as long as they are able to function in yeast. In order for promoters to be able to function in yeast, a multicopper oxidase mutant gene needs to be able to be transcribed therein. Examples of promoters include, but are not particularly limited to, a glyceraldehyde-3-phosphate dehydrogenase gene (TDH3) promoter, a 3-phosphoglycerate kinase gene (PGK1) promoter, and a high osmolarity response 7 gene (HOR7) promoter. In particular, a pyruvate decarboxylase enzyme gene (PDC1) promoter is preferable because it is highly capable of causing high expression of a downstream multicopper oxidase mutant gene.

In the present invention, an expression vector into which the multicopper oxidase mutant gene has been expressibly incorporated is introduced into a host by a conventional method so as to produce the multicopper oxidase mutant. Examples of a method that can be used as a method for introducing an expression vector into a host include, but are not limited to, a variety of conventionally known methods such as an electroporation method (Meth. Enzym., 194, p. 182 (1990)), a spheroplast method (Proc. Natl. Acad. Sci. USA, 75, p. 1929 (1978)), and a lithium acetate method (J. Bacteriology, 153, p. 163 (1983), Proc. Natl. Acad. Sci. USA, 75 p. 1929 (1978), Methods in yeast genetics, 2000 Edition: A Cold Spring Harbor Laboratory Course Manual). When a protein production system in which Escherichia coli serves as a host is used, a conventionally used expression vector comprising a multicopper oxidase mutant gene can be introduced into Escherichia coli. Typically, a vector has selection marker genes, cloning sites, and expression control regions (promoters and terminators). Such vector is well known in the art and commercially available. Examples of vectors that can be used include Escherichia coli-derived plasmids (e.g., ColE plasmids such as pBR322, pBR325, pUC18, pUC19, pUC119, pTV118N, pTV119N, pBluescript, pHSG298, pHSG396, and pTrc99A; p1A plasmids such as pACYC177 and pACYC184; and pSC101 plasmids such as pMW118, pMW119, pMW218, and pMW219) and Bacillus subtilis-derived plasmids (e.g., pUB110 and pTP5). Further, examples of phage DNA that can be used include lambda phages (e.g., Charon4A, Charon21A, EMBL3, EMBL4, lambda gt100, gt11, and zap), phi X174, M13mp18, and M13mp19. In addition to Escherichia coli, Bacillus subtilis can be used as a host.

Further, when a cell-free protein production system is used, an appropriate expression vector comprising a multicopper oxidase mutant gene is used in the system. An example of a cell-free protein production system that can be used is a cell extract obtained by disrupting Escherichia coli, wheat germ extract/rabbit reticulocyte lysate, or the like and removing membrane components via centrifugation. Also, a cell-free protein production system referred to as a so-called “PURE” system can be used.

In any case, a multicopper oxidase mutant can be purified by a conventional method in a protein production system using yeast, Escherichia coli, or the like or in a cell-free protein production system. For purification of a multicopper oxidase mutant, the following techniques can be used alone or in combination: affinity chromatography, ion-exchange chromatography, hydrophobic interaction chromatography, ethanol precipitation, reverse-phase HPLC, silica chromatography, cation-exchange resin (e.g., DEAE) chromatography, chromatofocusing, SDS-PAGE, ammonium sulfate precipitation method, and gel filtration.

<Mode of Using Multicopper Oxidase Mutants>

The above multicopper oxidase mutant can be used as an excellent alternative to a multicopper oxidase in any conventional reaction system. In particular, the above multicopper oxidase mutant has excellent resistance to imidazole compounds compared with the corresponding unmutated multicopper oxidase. Therefore, the above multicopper oxidase mutant is preferably used in a conventional reaction system, for which a multicopper oxidase has been used in combination with imidazole compounds. For instance, the multicopper oxidase mutant can be used for cathode electrodes for fuel cells. In this case, fuel cells preferably contain imidazole compounds as electrolytes. In general, when a multicopper oxidase mutant is used as a cathode electrode, a multicopper oxidase mutant can be immobilized to a material (e.g., porous carbon material) to be used as an electrode.

In addition, the multicopper oxidase mutant can be used for any type of fuel cell regardless of fuel cell configuration or structure. An example of a fuel cell is a fuel cell having a structure in which a cathode and an anode face each other separated by an electrolyte. An electrolyte used herein is not particularly limited. However, an electrolyte comprising imidazole compound(s) is preferable. This is because an electrolyte comprising imidazole compound(s) has excellent battery characteristics. Examples of imidazole compounds used herein include imidazole, triazole, a pyridine derivative, a bipyridine derivative, an imidazole derivative (e.g., histidine, 1-methylimidazole, 2-methylimidazole, 4-methylimidazole, 2-ethylimidazole, imidazole-2-ethylcarboxylate, imidazole-2-carboxaldehyde, imidazole-4-carboxylate, imidazole-4,5-dicarboxylate, imidazole-1-yl-acetate, 2-acetylbenzimidazole, 1-acetylimidazole, N-acetylimidazole, 2-aminobenzimidazole, N-(3-aminopropyl)imidazole, 5-amino-2-(trifluoromethyl)benzimidazole, 4-azabenzimidazole, 4-aza-2-mercaptobenzimidazole, benzimidazole, 1-benzylimidazole, or 1-butylimidazole).

In addition, examples of fuel available for fuel cells include polysaccharides (e.g., an oligosaccharide such as a disaccharide, trisaccharide, or tetrasaccharide) and monosaccharides. When a polysaccharide is used, it is preferable to use a degradative enzyme that promotes degradation such as hydrolysis of a polysaccharide, and produces a monosaccharide such as glucose in combination therewith. Specific examples of polysaccharides include starch, amylose, amylopectin, glycogen, cellulose, maltose, sucrose, and lactose. Such polysaccharide is formed as a result of the biding of two monosaccharides. Any polysaccharide comprises glucose as a monosaccharide, which is a sugar-binding unit.

### EXAMPLES

The present invention is hereafter described in greater detail with reference to the following examples, although the technical scope of the present invention is not limited thereto.

### Example 1

(1) Construction of a Library to be Screened

Three fragments were prepared by the primary PCR described below and ligated by the secondary PCR so as to construct a library.

1-1. Primary PCR

1-1-1. Error-Prone PCR

PCR was performed under the conditions listed below using a Diversify PCR Random Mutagenesis Kit (Clontech) for random mutation of the B. subtilis-derived multicopper oxidase gene (BOD gene). Table 2 lists the sequences of primers used. In addition, the template DNA used was obtained by cloning the B. subtilis-derived BOD gene into a pET23b(+) vector (NdeI/XhoI site). The nucleotide sequence of the B. subtilis-derived BOD gene is shown in SEQ ID NO: 1. The amino acid sequence of a multicopper oxidase encoded by the BOD gene is shown in SEQ ID NO: 2.

TABLE 1

| | | |
|---|---|---|
| H₂O | 39.5 | μl |
| 10XTITANIUM Taq Buffer | 5 | μl |
| dGTP (2 mM) | 1 | μl |
| 50XDiversify dNTP Mix | 0.5 | μl |
| primer1 (10 pmol/μl) | 1 | μl |
| primer2 (10 pmol/μl) | 1 | μl |
| Template DNA | 1 | μl |
| TITANIUM Taq Polymerase | 1 | μl |
| Total | 50 | μl |

TABLE 2

| Primer | Name | Sequence (5′ → 3′) |
|---|---|---|
| Primer 1 | bsEP5F | CTTTAAGAAGGAGATATACATAATG |
| Primer 2 | bsEP3Rstp | GGTGGTGGTGGTGCTCGAGTTA |

The following PCR reaction cycles were used: 94 degrees C. for 30 seconds; 25 cycles of 94 degrees C. for 30 seconds, 55 degrees C. for 30 seconds, and 68 degrees C. for 1.5 minutes; 68 degrees C. for 1 minute; and 4 degrees C. (for an indefinite period). The obtained PCR product was subjected to agarose electrophoresis. Then, bands were excised and purified according to a conventional method.

1-1-2. Preparation of PCR Fragments Comprising Vectors

Fragments 1 and 2 were prepared by PCR using the reaction solution compositions listed in tables 3 and 4. The enzyme used herein was KOD-Plus-DNA Polymerase. Table 5 shows the sequences of primers used herein.

TABLE 3
<Fragment 1>

| | | |
|---|---|---|
| 10xBuffer | 5 | ul |
| 2 mM dNTP | 5 | ul |
| 25 mM MgSO₄ | 2 | ul |
| primer: bsHomoLinkF-BglII (10 pmol/ul) | 1.5 | ul |
| primer: bsEP5R (10 pmol/ul) | 1.5 | ul |
| Template DNA (the same as used in 1-1-1.) | 1 | ul |
| KOD Plus polymerase | 1 | ul |
| dH₂O | 33 | ul |
| Total | 50 | ul |

TABLE 4
<Fragment 2>

| | | |
|---|---|---|
| 10xBuffer | 5 | ul |
| 2 mM dNTP | 5 | ul |
| 25 mM MgSO₄ | 2 | ul |
| primer: bsEP3Fstp (10 pmol/ul) | 1.5 | ul |
| primer: bsHomoLinkR (10 pmol/ul) | 1.5 | ul |
| Template DNA (the same as used in 1-1-1.) | 1 | ul |
| KOD Plus polymerase | 1 | ul |
| dH₂O | 33 | ul |
| Total | 50 | ul |

TABLE 5

| Name | Sequence (5′ → 3′) |
|---|---|
| bsHomoLinkF- | CTACGAGAGCCTACGGTTTACCACTCAGAT |
| BglII | CTCGATCCCGCGAAATTAAT |
| bsEP5R | CATTATGTATATCTCCTTCTTAAAG |
| bsEP3Fstp | TAACTCGAGCACCACCACCACC |
| bsHomoLinkR | CTACGAGAGCCTACGGTTTACCACTCTCCGGAT |
| | ATAGTTCCTCCTTTCAG |

The following PCR reaction cycles were used: 94 degrees C. for 2 minutes; 30 cycles of 94 degrees C. for 15 seconds, 55 degrees C. for 30 seconds, and 68 degrees C. for 1 minute; 68 degrees C. for 5 minutes; and 4 degrees C. (for an indefinite period). The obtained PCR product was subjected to agarose electrophoresis. Then, bands were excised and purified according to a conventional method.

1-2. Secondary PCR

PCR was performed using the reaction solution composition listed in table 6 for ligation of the three fragments prepared in 1-1.

TABLE 6

| | | |
|---|---|---|
| 10xBuffer | 5 | ul |
| 2 mM dNTP | 5 | ul |
| 25 mM MgSO₄ | 2 | ul |
| primer: bsLinkerFR (10 pmol/ul) | 3 | ul |
| 1-1-1. fragment | 1 | μl |
| 1-1-2. fragment 1 | 1 | μl |
| 1-1-2. fragment 2 | 0.5 | μl |
| KOD Plus polymerase | 1 | ul |
| dH₂O | 31.5 | ul |
| Total | 50 | ul |

The following was used as the PCR primer: bsLinkerFR: 5′-TACAATACTAATCTACGAGAGCCTACGGTTTACCACTC-3′. The following PCR reaction cycles were used: 94 degrees C. for 2 minutes; 30 cycles of 94 degrees C. for 15 seconds, 53 degrees C. for 30 seconds, and 68 degrees C. for 2 minutes; 68 degrees C. for 5 minutes; and 4 degrees C. (for an indefinite period). The obtained PCR product was confirmed to have a single amplified band by agarose electrophoresis. The band was excised and purified according to a conventional method. The eluate was designated as a library.

(2) Recombinant BOD Screening

2-1. PCR

The library solution was diluted. PCR was performed using the reaction solution composition listed in table 7.

TABLE 7

| | | |
|---|---|---|
| dH₂O | 4.36 | ul |
| 5.7M betaine | 2 | ul |
| 10xBuffer | 1 | ul |
| 2 mM dNTP | 1 | ul |
| 25 mM MgSO₄ | 0.4 | ul |
| primer: bsHomo (100 pmol/ul) | 0.04 | ul |
| KOD Plus (1 U/μl) | 0.2 | ul |
| Library solution | 1 | ul |
| Total | 10 | ul |

The following was used as the PCR primer bsHomo: 5′-CTACGAGAGCCTACGGTTTACCACTC-3′. The following PCR reaction cycles were used: 94 degrees C. for 2 minutes; 40 cycles of 94 degrees C. for 20 seconds and 68 degrees C. for 2 minutes; and 4 degrees C. (for an indefinite period).

2-2. Synthesis of Recombinant BOD in the Cell-Free Translation System

Translation was carried out using the Escherichia coli B strain-derived S30 fraction under the conditions described below. Table 8 lists the reaction solution composition.

TABLE 8

| | | |
|---|---|---|
| S30 | 5 | μl |
| 50 mM NTP | 0.2 | μl |
| 1M HEPES-KOH | 0.55 | μl |
| 3M potassium glutamate | 0.7 | μl |
| 2M ammonium acetate | 0.14 | μl |
| 20 mg/ml tRNA | 0.08 | μl |
| 1 mg/ml rifampicin | 0.08 | μl |
| 5M creatine phosphate | 0.08 | μl |
| 12 mg/ml creatine kinase | 0.15 | μl |
| Amino acid mix (*) | 0.3 | μl |
| 100 mM cAMP | 0.4 | μl |
| 5.7M betaine | 1.2 | μl |
| 60% PEG6000 | 0.6 | μl |
| 44.5 mM Mg(OAc)₂ | 0.5 | μl |
| H₂O | 0.14 | μl |
| PCRproduct | 1 | μl |
| Total | 11.12 | μl |

Table 9 shows the composition of the amino acid mixture listed in table 8.

TABLE 9

| | | |
|---|---|---|
| RPM1 1640 AMINO ACID SOLUTION 50X (SIGMA) | 800 | μl |
| 50 mM glutamine (Kyowa Hakko Industry) | 100 | μl |
| 50 mM alanine (Kyowa Hakko Industry) | 100 | μl |
| Total | 1 | ml |

A translation reaction took place at 25 degrees C. for 1.5 hours in a thermal cycler. Subsequently, the translation product was mixed with a 2 mM Cu/50 mM HEPES-KOH (pH 7.5) solution (11.12 microliters) and left overnight at 4 degrees C. for copper adsorption.

2-3. Activity Determination

2-3-1. Initial Activity

The BOD activity in the reaction solution was determined under the conditions described below. Table 10 lists the reaction solution composition.

TABLE 10

| | | |
|---|---|---|
| Mcllvaine buffer (pH 5) | 140.5 | μl |
| 20 mM ABTS | 7.5 | μl |
| Translation reaction solution | 2 | μl |
| Total | 150 | μl |

In addition, a reaction was carried out under conditions comprising heating at 37 degrees C. for 40 minutes in a water tank preliminarily heated to 37 degrees C. Then, the occurrence or nonoccurrence of coloration was observed.

2-3-2. Confirmation of Resistance to Imidazole

Each sample for which coloration had been observed in 2-3-1 was examined for improvement of resistance to imidazole using the following procedures. Table 11 shows the reaction solution composition.

TABLE 11

| | | |
|---|---|---|
| 3M imidazole (pH7.0) | 1.17 | μl |
| H₂O | 4.83 | μl |
| Translation reaction solutionlid | 1 | μl |
| Total | 7 | μl |

The reaction solution with the above composition was heated at 90 degrees C. for 30 minutes in a thermal cycler. Thereafter, an activity determination reaction solution (143 microliters) was added. Table 12 lists the composition of the activity determination reaction solution.

TABLE 12

| | | |
|---|---|---|
| Mellvaine buffer (pH 5) | 135.5 | μl |
| 20 mM ABTS | 7.5 | μl |
| Total | 143 | μl |

The solution supplemented with the activity determination reaction solution was heated in a thermal cycler at 37 degrees C. for 20 minutes. After heating, coloration was macroscopically observed.

(3) Evaluation of Mutants Extracted by Screening

3-1. Cloning of a Promising Mutant

Each mutant extracted in 2-3-2 was cloned into a pET23b(+) vector.

3-2. Evaluation

3-2-1. Preparation of Template DNA

PCR was performed under the conditions described below. Table 13 lists the reaction solution composition. Table 14 shows the sequences of primers used herein.

TABLE 13

| | | |
|---|---|---|
| 10xBuffer | 5 | μl |
| 2 mM dNTP | 5 | μl |
| 25 mM MgSO₄ | 2 | μl |
| primer: bsHomoLinkF-BglII (10 pmo/μl) | 1.5 | μl |
| primer: bsHomoLinkR (10 pmo/μl) | 1.5 | μl |
| KOD Plus polymerase | 1 | μl |
| Plasmid cloned in 3-1. | 0.3 | μl |
| dH₂O | 33.7 | μl |
| Total | 50 | μl |

TABLE 14

| Name | Sequence (5′ → 3′) |
|---|---|
| bsHomoLinkF-BglII | CTACGAGAGCCTACGGTTTACCACTCAGATC |
| | TCGATCCCGCGAAATTAAT |
| bsHomoLinkR | CTACGAGAGCCTACGGTTTACCACTCTCCGG |
| | ATATAGTTCCTCCTTTCAG |

The following PCR reaction cycles were used: 94 degrees for 2 minutes; 25 cycles of 94 degrees C. for 15 seconds, 53 degrees C. for 30 seconds, and 68 degrees C. for 2 minutes; 68 degrees C. for 2 minutes; and 4 degrees C. (for an indefinite period). The PCR reaction solution was purified using a MinElute PCR Purification Kit (QIAGEN). Thus, template DNA was obtained.

3-2-2. Synthesis of Recombinant BOD in the Cell-Free Translation System

Synthesis was carried out in the same manner as that used in 2-2.

3-2-3. Activity Determination

3-2-3-1. Initial Activity

The BOD activity in the reaction solution was determined under the conditions described below. Table 15 shows the reaction solution composition.

TABLE 15

| | | |
|---|---|---|
| Mcllvaine buffer (pH 5) | 140.5 | μl |
| 20 mM ABTS | 7.5 | μl |
| Translation reaction solution | 2 | μl |
| Total | 150 | μl |

A reaction took place in the reaction solution in a thermal cycler at 37 degrees C. for 20 minutes, followed by measurement of A420 using infinite M200 (TECAN).

3-2-3-2. Confirmation of Resistance to Imidazole

The improvement of resistance to imidazole was evaluated by the procedures described below. Table 16 shows the reaction solution composition.

TABLE 16

| | | |
|---|---|---|
| 3M imidazole HCl (pH7.0) | 1.17 | μl |
| H₂O | 4.83 | μl |
| Translation reaction solution | 1 | μl |
| Total | 7 | μl |

The reaction solution with the above composition was heated in a thermal cycler at 90 degrees C. for 30 minutes. Thereafter, an activity determination reaction solution (143 microliters) was added. Table 17 lists the composition of the activity determination reaction solution.

TABLE 17

| | | |
|---|---|---|
| Mcllvaine buffer (pH 5) | 135.5 | μl |
| 20 mM ABTS | 7.5 | μl |
| Total | 143 | μl |

A reaction took place in the reaction solution in a thermal cycler at 37 degrees C. for 20 minutes, followed by measurement of A420 using infinite M200 (TECAN). FIG. 2 shows the results. In addition, the nucleotide sequences of the individual mutants were identified using a sequencer by a conventional method. As shown in FIG. 2, a multicopper oxidase mutant (M157L) obtained by substituting methionine at position 157 in the amino acid sequence of a multicopper oxidase (encoded by the B. subtilis-derived BOD gene) with leucine was found to have excellent resistance to imidazole compounds. Similarly, a multicopper oxidase mutant (P414L or P414T) obtained by substituting proline at position 414 in the above amino acid sequence with leucine or threonine was found to have excellent resistance to imidazole compounds. In addition, a multicopper oxidase mutant having the M157L and P414L substitution mutations was found to have even more excellent resistance to imidazole compounds. Interestingly, a multicopper oxidase mutant (H90R) obtained by substituting histidine at position 90 in the amino acid sequence of the wild-type multicopper oxidase with arginine was comparable to the wild-type multicopper oxidase in terms of resistance to imidazole compounds. However, it was found that the H90R mutation itself enhances the resistance to imidazole compounds improved as a result of the M157L mutation. That is, it was revealed that a multicopper oxidase mutant having the H90R and M157L mutations has remarkably improved resistance to imidazole compounds over a multicopper oxidase mutant having the M157L mutation alone.

As shown in FIG. 2, a variety of mutants were identified as a result of single substitution mutations. For example, a mutation of a multicopper oxidase via substitution of methionine at position 335 in the amino acid sequence with valine resulted in a multicopper oxidase mutant (M335V). In this case, however, effects for improving resistance to imidazole compounds were not confirmed. Further, no mutation which causes a substitution of an amino acid at a different position so as to enhance the improved resistance to imidazole compounds was observed, in addition to the H90R mutation.

FIG. 3 shows the results of a comparison of BOD activity (converted to specific activity) among different multicopper oxidase mutants shown in FIG. 2. As shown in FIG. 3, a multicopper oxidase mutant having P414L or P414T was found to have very high specific activity. As in the case of such multicopper oxidase mutant having P414L or P414T, it was revealed in the Examples that there are substitution mutations that improve not only the resistance to imidazole compounds but also enzyme activity.

1. A multicopper oxidase mutant, which comprises an amino acid sequence derived from the amino acid sequence of a multicopper oxidase shown in SEQ ID NO: 2 by substitution of either or both an amino acid residue corresponding to methionine at position 157 and an amino acid residue corresponding to proline at position 414 with a different amino acid and has activity of catalyzing a reaction generating water molecules via four-electron reduction of oxygen molecules using ABTS (2,2′-azinobis(3-ethylbenzoline-6-sulfonate)) as a substrate.

2. The multicopper oxidase mutant of claim 1, in which the amino acid residue corresponding to methionine at position 157 has been substituted with leucine.

3. The multicopper oxidase mutant of claim 1, in which the amino acid residue corresponding to proline at position 414 has been substituted with leucine or threonine.

4. The multicopper oxidase mutant of claim 1, which is a mutant of a Bacillus subtilis-derived multicopper oxidase.

5. The multicopper oxidase mutant of claim 2, in which an amino acid residue corresponding to histidine at position 90 in the multicopper oxidase comprising the amino acid sequence shown in SEQ ID NO: 2 has been substituted with a different amino acid.

6. A gene encoding the multicopper oxidase mutant of claim 1.

7. A biofuel cell comprising the multicopper oxidase mutant of claim 1 as a cathode electrode.

8. The biofuel cell of claim 7, which has a structure in which a cathode electrode and an anode electrode face each other separated by an electrolyte.

9. The biofuel cell of claim 8, wherein the electrolyte comprises imidazole compounds.

10. The gene encoding the multicopper oxidase mutant of claim 6, in which the amino acid residue corresponding to methionine at position 157 has been substituted with leucine.

11. The gene encoding the multicopper oxidase mutant of claim 6, in which the amino acid residue corresponding to proline at position 414 has been substituted with leucine or threonine.

12. The gene encoding the multicopper oxidase mutant of claim 6, which the multicopper oxidase mutant is a mutant of a Bacillus subtilis-derived multicopper oxidase.

13. The gene encoding the multicopper oxidase mutant of claim 6, in which an amino acid residue corresponding to histidine at position 90 in the multicopper oxidase comprising the amino acid sequence shown in SEQ ID NO: 2 has been substituted with a different amino acid.

14. The biofuel cell of claim 7, in which the amino acid residue corresponding to methionine at position 157 has been substituted with leucine.

15. The biofuel cell of claim 7, in which the amino acid residue corresponding to proline at position 414 has been substituted with leucine or threonine.

16. The biofuel cell of claim 7, which the multicopper oxidase mutant is a mutant of a Bacillus subtilis-derived multicopper oxidase.

17. The biofuel cell of claim 7, in which an amino acid residue corresponding to histidine at position 90 in the multicopper oxidase comprising the amino acid sequence shown in SEQ ID NO: 2 has been substituted with a different amino acid.


SEQUENCE LISTING (4 sequences)

SEQ ID NO: 1 - DNA, 1536 bp, Bacillus subtilis
  CDS (1)..(1536)
atgacacttgaaaaatttgtggatgctctcccaatcccagatacactaaagccagtacagcaatcaaaagaaaaaacatactacgaagtcaccatggaggaatgcactcatcagctccatcgcgatctccctccaacccgcctgtggggctacaacggcttatttccgggaccgaccattgaggttaaaagaaatgaaaacgtatatgtaaaatggatgaataaccttccttccacgcatttccttccgattgatcacaccattcatcacagtgacagccagcatgaagagcccgaggtaaagactgttgttcatttacacggcggcgtcacgccagatgatagtgacgggtatccggaggcttggttttccaaagactttgaacaaacaggaccttatttcaaaagagaggtttatcattatccaaaccagcagcgcggggctatattgtggtatcacgatcacgccatggcgctcaccaggctaaatgtctatgccggacttgtcggtgcatatatcattcatgacccaaaggaaaaacgcttaaaactgccttcagacgaatacgatgtgccgcttcttatcacagaccgcacgatcaatgaggatggttctttgttttatccgagcgcaccggaaaacccttctccgtcactgcctaatccttcaatcgttccggctttttgcggagaaaccatactcgtcaacgggaaggtatggccatacttggaagtcgagccaaggaaataccgattccgtgtcatcaacgcctccaatacaagaacctataacctgtcactcgataatggcggagattttattcagattggttcagatggagggctcctgccgcgatctgttaaactgaattctttcagccttgcgcctgctgaacgttacgatatcatcattgacttcacagcatatgaaggagaatcgatcattttggcaaacagcgcgggctgcggcggtgacgtcaatcctgaaacagatgcgaatatcatgcaattcagagtcacaaaaccattggcacaaaagacgaaagcagaaagccgaagtacctcgcctcatacccttcggtacagcatgaaagatacaaacatcagaacgttaaaactggcaggcacccaggacgaatacggcagacccgtccttctgcttaataacaaacgctggcacgatcccgtcacagaaacaccaaaagtcggcacaactgaaatatggtccattatcaaccgacacgcggaacacatcctgatccacctgcatctagtctccttccgtgtattagaccggcggccgtttgatatcgcccgttatcaagaaagcggggaattgtcctatacagtccgctgtcccgccgccgcaagtgaaaagggctggaaagacaccattcaagcgcatgcaggtgaagtcctgagaatcgcggcgacattcggtccgtacagcggacgatacgtatggcattgccatattctagagcatgaagactatgacatgatgagaccgatggatataactgatccccataaataa

SEQ ID NO: 2 - protein, 511 aa, Bacillus subtilis
MTLEKFVDALPIPDTLKPVQQSKEKTYYEVTMEECTHQLHRDLPPTRLWGYNGLFPGPTIEVKRNENVYVKWMNNLPSTHFLPIDHTIHHSDSQHEEPEVKTVVHLHGGVTPDDSDGYPEAWFSKDFEQTGPYFKREVYHYPNQQRGAILWYHDHAMALTRLNVYAGLVGAYIIHDPKEKRLKLPSDEYDVPLLITDRTINEDGSLFYPSAPENPSPSLPNPSIVPAFCGETILVNGKVWPYLEVEPRKYRFRVINASNTRTYNLSLDNGGDFIQIGSDGGLLPRSVKLNSFSLAPAERYDIIIDFTAYEGESIILANSAGCGGDVNPETDANIMQFRVTKPLAQKTKAESRSTSPHTLRYSMKDTNIRTLKLAGTQDEYGRPVLLLNNKRWHDPVTETPKVGTTEIWSIINRHAEHILIHLHLVSFRVLDRRPFDIARYQESGELSYTVRCPAAASEKGWKDTIQAHAGEVLRIAATFGPYSGRYVWHCHILEHEDYDMMRPMDITDPHK

SEQ ID NO: 3 - protein, 572 aa, Myrothecium verrucaria
MFKHTLGAAALSLLFNSNAVQASPVPETSPATGHLFKRVAQISPQYPMFTVPLPIPPVKQPRLTVTNPVNGGEIWYYEVEIKPFTHQVYPDLGSADLVGYDGMSPGPTFQVPRGVETVVRFINNAEAPNSVHLHGSFSRAAFDGWAEDITEPGSFKDYYYPNRNSARTLWYHDHAMHITAENAYRGQAGLYMLTDPAEDALNLPSGYGEFDIPMILTSKQYTANGNLVTTNGELNSFWGDVIHVNGQPWPFKNVEPRKYRFRFLDAAVSRSFGLYFADTDAIDTRLPFKVIASDSGLLEHPADTSLLYISMAERYEVVFDFSDYAGKTIELRNLGGSIGGIGTDTDYDNTDKVMRFVVADPTTPPDTSVVPANLRDVPFPSPTTNTPRQFRFGRTGPTWTINGVAFADVQNRLLANVPVGTVERWELINAGNGWTHPIHIHLVDFKVISRTSGNNARTVMPYESGLKDVVWLGRRETVVVEAHYAPFKGVYMFHCHNLIHEDHDMMAAFNATVLPDYGYNATVFVDPMEELWQARPYELGEFQAQSGQFSVQAVTERIQTMAEYRPYAAADE

SEQ ID NO: 4 - protein, 534 aa, Myrothecium verrucaria
VAQISPQYPMFTVPLPIPPVKQPRLTVTNPVNGQEIWYYEVEIKPFTHQVYPDLGSADLVGYDGMSPGPTFQVPRGVETVVRFINNAEAPNSVHLHGSFSRAAFDGWAEDITEPGSFKDYYYPNRQSARTLWYHDHAMHITAENAYRGQAGLYMLTDPAEDALNLPSGYGEFDIPMILTSKQYTANGNLVTTNGELNSFWGDVIHVNGQPWPFKNVEPRKYRFRFLDAAVSRSFGLYFADTDAIDTRLPFKVIASDSGLLEHPADTSLLYISMAERYEVVFDFSDYAGKTIELRNLGGSIGGIGTDTDYDNTDKVMRFVVADDTTQPDTSVVPANLRDVPFPSPTTNTPRQFRFGRTGPTWTINGVAFADVQNRLLANVPVGTVERWELINAGNGWTHPIHIHLVDFKVISRTSGNNARTVMPYESGLKDVVWLGRRETVVVEAHYAPFPGVYMFHCHNLIHEDHDMMAAFNATVLPDYGYNATVFVDPMEELWQARPYELGEFQAQSGQFSVQAVTERIQTMAEYRPYAAADE